(19) United States
(12) Patent Application Publication
(10) Pub. No.: US 2004/0117028 A1
(43) Pub. Date: Jun. 17, 2004

(54) Method and device for providing information after insertion of a prosthesis in a hip-joint

(76) Inventor: Bjorn Franc Iversen, Humlabaek (DK)

Correspondence Address: SIERRA PATENT GROUP, LTD., P O BOX 6149, STATELINE, NV, 89449, US

(21) Appl. No.: 10/471,851

(22) PCT Filed: Mar. 12, 2002
(86) PCT No.: PCT/NO02/00102

(30) Foreign Application Priority Data
Mar. 13, 2001 (NO) 2001 1275

(51) Int. Cl. A61F 2/32

(52) U.S. Cl. 623/22.11; 623/22.12; 623/908

(57) ABSTRACT

Method an device for providing information after insertion of a prosthesis in a hip joint comprising prosthesis stem, and a prosthesis cup and based on this information the position of the components of a hip prosthesis is provided. The prosthesis stem and the prosthesis cup or the part of the body in which the prosthesis is provided can include markers that can be shown on an X-ray picture. The X-ray picture is downloaded in an image processing program having assistance lines for determining the position of the prosthesis components. The X-ray picture is downloaded automatically or manually in the image processing program. The assistance lines are brought into coincidence with the images of the markers on the X-ray picture. Thereafter a calculation unit in the image processing unit calculates the angles for the respective prosthesis components.

### CROSS-REFERENCE TO RELATED APPLICATIONS

This application claims filing date priority benefit to Norwegian application no. 2001 1275 filed Mar. 13, 2001, and to PCT application no. PCT/NO02/00102, filed Mar. 12, 2002, under 35 U.S.C. 119 which is explicitly incorporated by reference as if set forth below.

### BACKGROUND

The present invention regards a method of and device for providing information regarding the result of inserting a prosthesis in a hip joint, and on the basis of this information indicate the position of the components of a femoral prosthesis, in order to be able to advise a patient with regard to what movements can be carried out without risking the prosthesis dislocating (luxating).

A femoral prosthesis consists of two main components, a prosthesis stem (the femoral component) and a cup (the acetabulum component). At one end, the. prosthesis stem is provided either with a spherical condyle or a prosthesis neck on which may be placed a condyle, where the condyle is designed for close, sliding accommodation in a spherical depression in the cup. Together, the prosthesis stem with the condyle and the cup will act as a ball and socket joint to replace the natural hip joint.

The other end of the prosthesis stem comprises an elongated part adapted to be attached to the hollowed femoral canal in the patient's femur.

The cup is adapted to be attached to the joint cavity on the patient's pelvis. The hemispherical depression in the cup is via a side face connected to an outer surface adapted to be attached to the pelvis. The outer surface may have various shapes, depending on how it is to be attached to the pelvis and any other choices made by the supplier. Many cups are shaped as an approximate hemisphere, where the outer surface of the hemisphere is adapted to be attached in the pelvis. The side face connecting the depression and the outer surface may be flat or optionally slope in towards the depression, which is preferably approximately central in the side face.

The prosthesis stem and the cup may be fixed to the femur and the pelvis, respectively, by use of acrylic cement or by a cement-less force fit.

When replacing a worn out hip joint with a prosthesis, the femur head is replaced by the upper femur being cut and the femoral canal of the femur being hollowed out at the top in order to give room for the elongated prosthesis stem, which is either cemented into the femoral canal or force fitted.

The cavity on the pelvis is milled out to receive the cup, which is then fixed either by means of cement or a force fit.

If the condyle of the prosthesis is detachable, this is placed on the prosthesis stem before the condyle is placed in the cup; the joint is assembled by lifting the patient's leg up to a natural position and inserting the condyle in the hollow of the cup, whereupon the incision is closed.

Such a prosthesis should give the patient a mobility that approximates that which is provided by the natural joint. However, as the joint capsule etc. is weakened or possibly removed during surgery, it becomes possible for the patient to move the operated leg into positions that are outside of the natural mobility. This may cause the condyle to jump out of the cup (luxation). Moreover, it is important that a “natural” movement of the joint does not cause the patient to get in a situation in which the leg ends up in positions where the neck of the prosthesis rides on the edge of the cup, as this may cause luxation. This happens through simple leverage. Luxation occurs in the case of between 2 and 9% of all patients who have had a femoral prosthesis put in. If this happens, the patient must be anaesthetized before the joint is put back into place. Some patients must have a new operation. The risk of luxation is much greater in patients whose prosthesis components are assembled so as to have an incorrect mutual positioning, than in those where the mutual positioning of the components is correct, as incorrectly assembled prosthesis components may result in the leverage effect as described above.

### SUMMARY

The present invention aims to provide a tool that makes it easier for the surgeon, following a prosthesis operation, to form a picture of the positioning of the prosthesis components, and thereby any tendency towards luxation. In possession of such information, the surgeon may after surgery give patients individual advice regarding which movements the new prosthesis joint will allow without risk of the prosthesis dislocating.

In orthopedic publications, it is opined than an optimum mutual relationship between the prosthesis stem and the cup results in a reduced risk of luxation because the patient can go through the everyday natural range of motion (ROM) without the parts of the prosthesis ending up in such mutual positioning so as to entail a risk of luxation.

The inventor has previously, in experimental studies, shown (not published) that the most expedient ROM is achieved by assembling both prosthesis components in a manner so as to give them a forward angle of about 15 degrees relative to the frontal plane of the body, while the cup forms an angle of 45 degrees with the horizontal plane. In medical terminology, forward angling is termed anteversion.

The inventor has also previously shown (not published) that even though the optimum is to have each of the components angled forwards at 15 degrees, the result is almost as good if the sum of the forward angling of the two components is 30 degrees. Thus a prosthesis joint where the cup is angled forwards at 5 degrees and the prosthesis stem is angled forwards at 25 degrees will result in an ROM for the patient that is nearly as expedient as if both components were angled forward at 15 degrees, the sum of the forward angling in both cases being 30 degrees.

In a previous application, the inventor has described methods and devices for ensuring the above mentioned correct positioning of the prosthesis components. However it is desirable, regardless of the method used, to be able to perform a quality check on the position of the prosthesis components, which moreover allows individual counseling of the patient with regard to which movements are considered detrimental.

### DESCRIPTION OF THE DRAWINGS

In the following, the invention will be described in greater detail with reference to the drawings, in which:

FIG. 1 shows the pelvic components (the cup) with a potential design of markers for known prosthesis positions.

FIG. 2 shows the femoral component (the prosthesis stem) with a design of markers for known positions relative to the geometry of the prosthesis.

FIG. 3 shows an alternative for fixing the patient's feet in a standardized and reproducible position when taking X-rays.

FIG. 4 shows an example of an X-ray following an operation.

FIG. 5 shows the display of a program for determining the position of the prosthesis after an operation.

### DETAILED DESCRIPTION

In order to be able to see the position of a femoral prosthesis on an X-ray, or for an image processing system to be able to determine the position of the femoral prosthesis, markers are required on, or in a known relation to, the various prosthesis components.

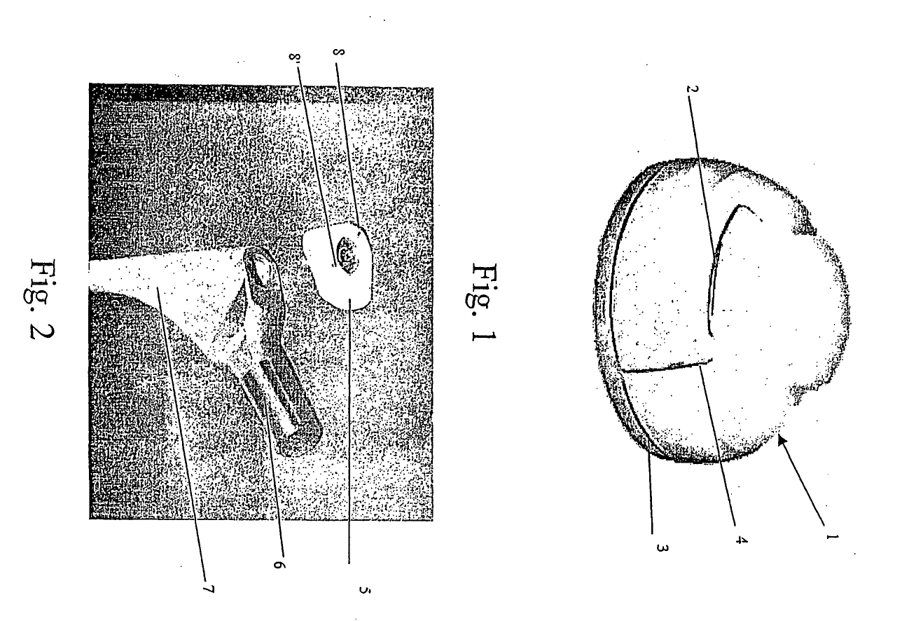

FIGS. 1 and 2 show examples of markers arranged on the pelvic component (the cup) and the femoral component (the prosthesis stem), respectively. FIG. 1 shows metal (wire) elements 2, 4, 3 of a known length and shape arranged on the outside of the prosthesis (the cup) 1. In the example shown, the wire element is an elongated wire arranged as a hook 2 in part of the periphery of the upper portion of the cup, which continues in a part 4 that extends in a perpendicular direction to the periphery, and which at the lower portion of the cup extends across the entire periphery 3. The cup, which is normally made from a plastic material, is not visible on the X-ray, and as such the metal wire will appear in full on the X-ray despite the metal wire being attached to the cup e.g. through being covered by the plastic material at certain attachment points.

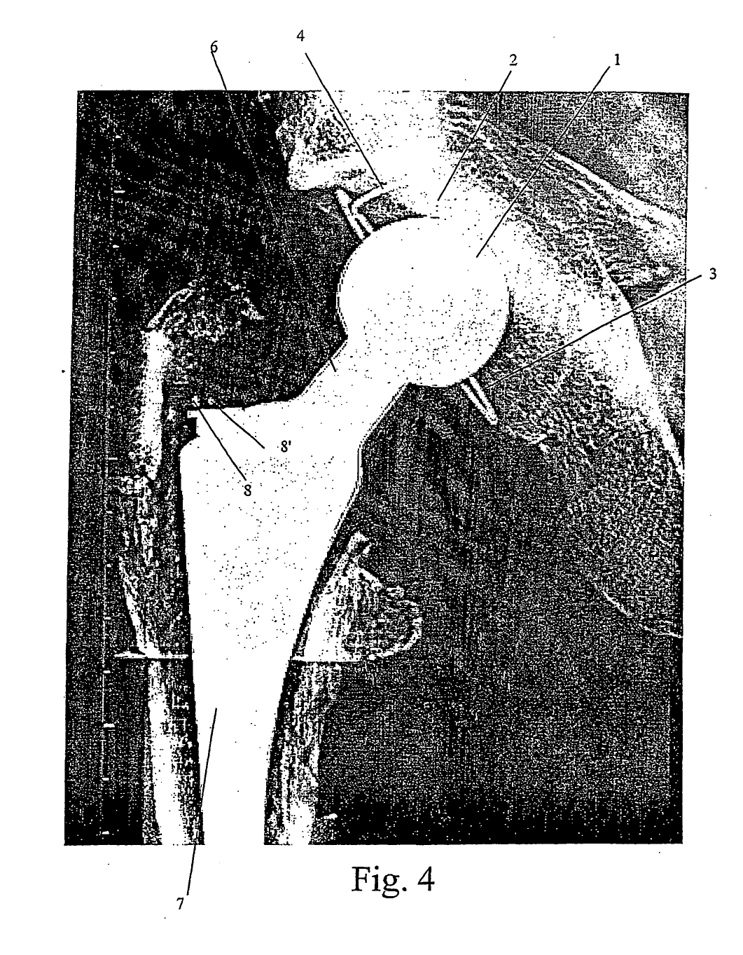

FIG. 4 shows an X-ray of a hip joint with an inserted prosthesis, indicating the markers 2, 3, 4 of the cup. The peripheral portion 3 defines an ellipse that varies from a line to a circle, depending on the position of the cup. Likewise, the length of the part 2 will vary according to the position of the cup 1.

This description of the invention uses this example of markers; however other forms of markers will be possible, such as those described e.g. in GB 2 134 360.

Another possibility is to drill holes in the cup, where small, X-ray proof markers are provided at known positions in the plastic part of the prosthesis.

Furthermore, metal elements may be provided, which are knocked or drilled into the pelvic bone at a known distance from and angle to the prosthesis. This may be achieved by means of a tool, e.g. a pair of tongs, which grips around the cup after the cup has been positioned and fixed in the joint socket on the pelvis. This tool is shaped so as to only fit the cup in one position. The tool is equipped with one or more guiding channels through which markers or components in which the markers are embedded, are screwed or knocked into the pelvic bone. Using one or more markers attached to the pelvic bone allows the use of standard prosthesis cups that do not require modification in order for the image processing program to be able to determine the angles of the prosthesis cup in the joint socket on the pelvis. The shape of the marker on the cup and the spatial positioning of the marker disposed in the pelvis will vary on an X-ray in dependence on how the prosthesis cup is positioned in the joint socket on the pelvis.

FIG. 2 shows an example of a marker for the femoral component (the prosthesis stem), which is used in the further description of the invention. Here, balls 8, 8′ of tantalum are arranged diagonally across from each other in a plastic sleeve 5 that is screwed to the prosthesis stem 7. The distance between these balls 8, 8′ will vary on an X-ray in dependence on the direction in which the prosthesis neck points, whether it points forwards or backwards relative to the transversal plane of the patient (is anteverted or retroverted).

The measurement markers may be fixed to the prosthesis component, as they are screwed to existing or new holes in the prosthesis.

Another possibility is for the measurement markers to be shaped as a short metal or plastic pipe stub (1 to 2 mm) that is fixed to the prosthesis component by the pipe stub being guided down over the prosthesis neck and fixed to the prosthesis neck with a set screw or through spring loading. The pipe stub is provided with X-ray proof markers in a known position relative to the geometry of the prosthesis.

Measurement markers may be mounted by being threaded onto the prosthesis stem from below and fixed by friction or screws. They may also be disposed in a thin plastic or methyl methacrylate tube that is threaded onto the prosthesis stem from below.

It will also be possible to provide metal elements in the actual femoral bone, which are knocked or drilled into the femoral bone at a known distance from and angle relative to the prosthesis. This presupposes the use of a provisional tool that is mounted on the femoral prosthesis (e.g. on the prosthesis neck) and has points of support against other areas of the prosthesis, so as to position the tool in a unique manner on the prosthesis. The tool may be provided with two or more guide channels through which markers are knocked into the femoral bone. Alternatively, it may hold a (plastic) block in which the markers are embedded, while the block is screwed to the bone.

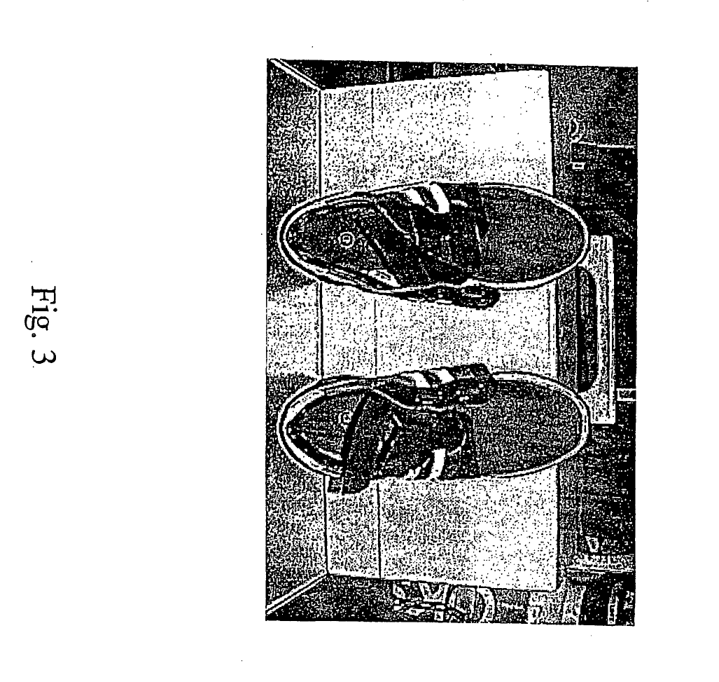

X-rays must be taken under standardized conditions. This is achieved by placing the patient in a reproducible position, through the feet being fixed in special shoes placed on an X-ray table as indicated in FIG. 3. Alternatively, the patient may lie on his back with his legs hanging off the edge of the X-ray table. This is commonly used, as the weight will then set the angle correctly. The patient is placed with his pelvis in the horizontal position, which is checked with a water level resting on the iliac crest (spina iliaca).

As previously mentioned, FIG. 4 shows an X-ray of a hip joint with an inserted prosthesis, where, in addition to the cup markers, markers 8,8′ also appear, and the distance between the markers gives a picture of the position of the prosthesis neck. Because the markers 8, 8′ (e.g. balls) have been placed diagonally in the plastic sleeve (plastic block) 5, an increasing distance between the balls 8, 8′ on an X-ray file will imply a decreasing anteversion or possibly retroversion, while a decreasing distance between the balls 8, 8′ will imply an increasing retroversion, i.e. a short distance means that the prosthesis neck is anteverted, and a great distance means that the prosthesis neck is retroverted.

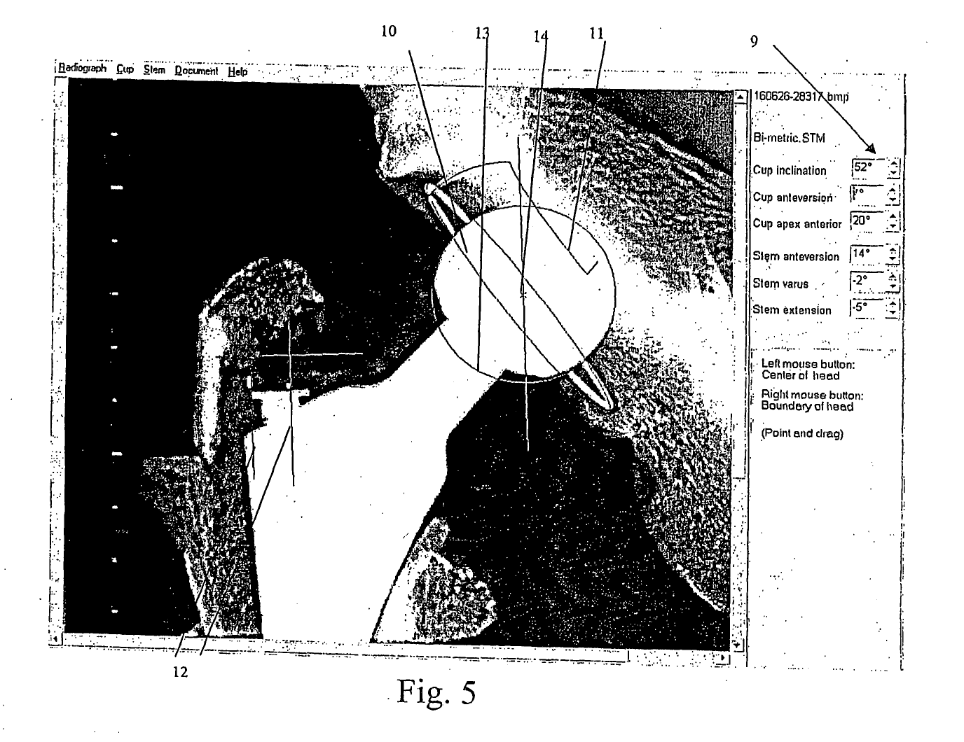

Based on the X-ray with the markers, the surgeon is therefore able, through manual measurement of the distances and angles, to make a statement regarding the position of the prosthesis. However, such a manual measurement is burdened with potential flaws and a not insignificant measuring uncertainty. Thus an automatic or partly automatic interpretation of the X-ray is desirable. This may be provided through. a computer program that superimposes auxiliary lines 10, 11, 12 (the display in FIG. 5) on the X-ray, which are guided over the respective markers, either manually by an operator for the computer processing equipment or automatically, by identification of the pixel value of the markers, and impose themselves over the markers. When the auxiliary lines have been arranged over the respective markers, a calculating unit in the image processing unit will calculate the angles on the basis of trigonometric principles, determining the number of degrees of anteversion or retroversion. The calculations may also be performed by means of looking up tables of angles that have been found through accurate measurements on models. Thus the surgeon will obtain a result that is reproducible and independent of the individual, and which will provide a statement on the quality of an operation, while allowing the surgeon to advise a patient as to which movements can be made without risking luxation (dislocating the prosthesis).

The image processing unit may be connected to the X-ray department, so that the X-ray may be transmitted directly to the image processing unit as a graphics file or be stored in a memory that may be accessed by the image processing unit, to allow the responsible surgeon to retrieve the X-ray into the image processing unit.

The image processing program may for instance be used as follows: The X-ray is retrieved into the program for calculating the orientation of the prosthesis stem and the cup, see FIG. 5. Upon retrieval, the program will request an answer to which hip the calculation is to apply; right or left. The program can also do this automatically if the X-ray is marked with an indication of side and the program automatically selects the correct calculation program on the basis of a pixel recognition function. Further, templates are selected for the above mentioned auxiliary lines for the relevant prosthesis type, cup and prosthesis stem, respectively. The templates will differ from one prosthesis to the next. These may be retrieved and superimposed on the X-ray automatically, based on the patient information that comes with the loading of the X-ray.

FIG. 5 shows a display of the X-ray with superimposed auxiliary lines adjusted and positioned correctly on the X-ray. This positioning may be done manually by operating the keyboard or mouse, e.g. by 15 depressing and holding the respective mouse buttons when the mouse arrow is located over circle 13 and dragging the circle 13 over the head of the prosthesis, or by the operator positioning the mouse arrow at the center of the head of the prosthesis and clicking on the left mouse button and positioning the mouse arrow on the outer edge of the circular head of the prosthesis and clicking on the right mouse button, whereupon the diameter of the circle 13 is reduced or increased, whereby correction is made for the degree of magnification on the X-ray film. In order to obtain coincidence with the outer edges of the head of the prosthesis, it may be necessary to repeat the process of centering the circle 13.

The auxiliary circle (the ellipse) 10 is then brought to coincide with the marker 3 (compare with FIG. 4, which is an X-ray of an inserted prosthesis). This is done by the operator adjusting the angles indicated on the display for cup inclination (inclination of the ellipse relative to the horizontal plane 10) and anteversion/retroversion (the degree of opening of the ellipse). The auxiliary line 11 is brought to coincide with the marker parts 4 and 2 by adjusting the apex of the cup (anterior posterior). The auxiliary lines 12 are then brought to run approximately vertically through the markers 8 and 8′ by adjusting the angles of stem varus, stem extension and stem anteversion (retroversion).

The auxiliary lines 12 are intersected by a line that is to be parallel to an approximately horizontal plane through the markers on the femoral component 12 by correct adjustment of the auxiliary lines. Following this setting of the auxiliary lines, the operator may read the spatial orientation of the prosthesis.

However these processes may, as indicated above, be carried out automatically by the program, by the image processing part of the program finding the co-ordinates of the auxiliary lines and positioning them correctly, thereby automatically providing the desired angles and information required by the operator and the surgeon in order to be able to prescribe the correct treatment and to advise the patient as to how to move in order to avoid luxation. An automatic solution would however require quality assurance through an operator approving the program results.

What is claimed is:

1. A method of controlling prosthesis positions in a body part where the prosthesis comprises a prosthesis stem and a prosthesis cup, where a selected number of markers is positioned on or by the prosthesis in a known mutual geometry configured to appear on an X-ray, the method comprising:
taking X-rays of the body part including the markers;
loading an X-ray image into an image processing program;
at least one of automatically defining and manually defining a number of artificial lines in the image processing program on the basis of the known mutual geometry of the measurement markers;
comparing the deviation between the positions of the artificial lines and the positions of the markers in the image processing program;
measuring the deviation between the positions of the artificial lines and the positions of the markers in the image processing program;
calculating the respective angles of the prosthesis components by use of a calculating unit in the image processing unit; and
displaying the values of the respective angles.

2. An arrangement at an image processing program for providing information regarding the result of inserting a prosthesis in a hip joint, the prosthesis including components such as a prosthesis stem and a prosthesis cup, wherein the prosthesis stem, prosthesis cup and body parts include markers configured to appear on an X-ray image, and based on information from the image the arrangement can determine position of the components of the prosthesis and body pars, the arrangement comprising:
an X-ray loading device configured to load an X-ray image into the image processing program;
an artificial line providing device configured to provide artificial lines for determining a position of the prosthesis components, wherein said artificial lines are designed to be brought to coincide with image of the markers on the X-ray; and
a calculating unit coupled to the image processing unit configured to calculate the respective angles of the prosthesis components in order to be displayed on a screen of a display unit.

3. The arrangement of claim 2 wherein said markers for the prosthesis stem are balls made from a material that will appear on an X-ray.

4. The arrangement of claim 3 wherein said markers comprise tantalum material arranged in plastic sleeves screwed into the prosthesis stem.

5. The arrangement of claim 2 wherein said markers for the prosthesis stem are formed by a pipe stub.

6. The arrangement of claim 5 wherein said pipe stub comprises one of a metal and a plastic.

7. The arrangement of claim 5 wherein said pipe stub is fixed to the prosthesis by the pipe stub being guided over a neck of the prosthesis.

8. The arrangement of claim 7 wherein said pipe stub is fixed to said neck selected from a group consisting of a set screw and spring loading.

9. The arrangement of claim 5 wherein said pipe stub is fixed to the prosthesis by the pipe stub being guided up over the prosthesis stem.

10. The arrangement of claim 5 wherein said pipe stub includes X-ray proof markers at known positions relative to the geometry of the prosthesis.

11. The arrangement of claim 2 wherein the markers for the prosthesis stem are formed by metal elements positioned in a femoral bone.

12. The arrangement of claim 11 wherein said markers are one of knocked into and drilled into said femoral bone at a known distance from an angle to the prosthesis.

13. The arrangement of claim 2 wherein said markers for the prosthesis cup are formed by means of a standard marker on the prosthesis cup, and at least one metal element marker is placed in a pelvic bone.

14. The arrangement of claim 13 wherein said at least one metal element marker is one of knocked and drilled into said pelvic bone at a known distance from an angle to the prosthesis cup.

15. The arrangement of claim 2 wherein the display unit is configured to display calculated angles and is configured to indicate the existence of one of a retroversion and an anteversion.
